(19) United States
(12) Patent Application Publication
(10) Pub. No.: US 2008/0171709 A1
(43) Pub. Date: Jul. 17, 2008

(54) Pharmaceutical Composition Comprising An Antiviral Agent, An Antitumoral Agent Or An Antiparasitic Agent, And An Active Agent Selected Among Carveol, Thymol, Eugenol, Borneol,And Carvacrol

(75) Inventor: Adnane Remmal, Fes (MA)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: ADVANCED SCIENTIFIC DEVELOPMENTS, Casablanca (MA)

(21) Appl. No.: 11/914,029

(22) PCT Filed: May 15, 2006
(86) PCT No.: PCT/IB06/01328

§ 371 Date: Jan. 22, 2008

(30) Foreign Application Priority Data
May 13, 2005 (IB) 2005/001314

(51) Int. Cl. A61K 31/704; A61K 31/05; A61P 35/00; A61P 33/00

(52) U.S. Cl. 514/34; 514/731; 514/733

(57) ABSTRACT

The invention relates to a pharmaceutical composition comprising at least one first active therapeutic substance selected among carveol, thymol, eugenol, borneol, carvacrol, alpha-ionone and beta-ionone, as well as isomers, derivatives and mixtures thereof, and comprising at least one second active therapeutic substance that is antitumoral. The invention is for use in the field of pharmaceutics.

The invention relates to a pharmaceutical composition comprising two therapeutically active substances one of which exerts a potentiating action on the other, and to the use of said composition.

It is known that the efficacy of therapeutic agents depends on the doses used which, in the case of partial resistance, necessitates increasing the doses of the therapeutic agents in order to attain the desired efficacy. This dose increase leads to problems with adverse effects and acute or chronic toxicity, which may considerably complicate the condition of the treated patients.

Said partial resistance may turn into complete resistance. In this case, increasing the dose no longer has any beneficial therapeutic effect; only the toxic effects are observed. The treatment in such a case consists in changing the therapeutic agent.

This chain of events can repeat itself and lead to the most serious situation: complete resistance to multiple therapeutic agents (multidrug resistance).

For instance, in particular, immunosuppressed patients become increasingly difficult to treat and their life expectancy is correspondingly shortened. Moreover, their quality of life is substantially affected by the administration of high doses of therapeutic agents.

The invention is directed at alleviating these problems by proposing to combine at least two therapeutically active substances, one of which potentiates the activity of the other, which not only makes it possible to lower the doses of each therapeutically active substance but also to treat patients afflicted with infections caused by resistant diseases.

In this regard, the invention provides a pharmaceutical composition characterized in that it comprises:

at least one first therapeutically active substance selected in the group consisting of carveol, thymol, eugenol, borneol, carvacrol, alpha-ionone, beta-ionone, and the isomers, derivatives and mixtures thereof,

and,

at least one second therapeutically active substance which is an antitumoral agent.

The first therapeutic substance can be obtained by chemical synthesis or from a plant source.

Preferably, the antitumoral agent in the composition of the invention is selected in the group consisting of folate antagonists, antimetabolites, alkylating agents, platinum salts, anthracyclin and intercalating agents, anti-topoisomerases, agents acting on cytoskeleton, bleomycin, asparaginase, and the mixtures thereof.

More preferably, the antitumoral agent is selected in the group consisting of methotrexate, 5-fluorouracil, fluorodeoxyuridine, cytosine arabinoside, 6-mercaptopurine, 6-thioguanine, mechloroethamine, cyclophosphamide, ifosfamide, melphalan, chlorambucil, thiotepa, mitomycin C, aziridinylbenzoquinone (AZQ), busulfan, carmustine (BCNU), lomustine (CCNU), fotemustine, carboplatin, daunorubicin, doxorubicin or adriamycin, epirubicin, dactinomycin or actinomycin D, mitoxanthrone, amsacrine, tenoposide, etoposide, irinotecan, topotecan, vincristine, vinblastine, vindesine, vinorelbine, taxol® (paclitaxel), taxotere® (docetaxel), and mixtures thereof.

A more particularly preferred antitumoral composition is a composition in which the first therapeutically active substance is carvacrol and the antitumoral agent is doxorubicin.

Another more particularly preferred antitumoral composition is a composition in which said first therapeutically active substance is thymol and the antitumoral agent is doxorubicin.

Yet another more particularly preferred antitumoral composition is a composition in which said first therapeutically active substance is carveol and the antitumoral agent is doxorubicin.

The invention also proposes a kit characterized in that it comprises at least one first container containing a first therapeutically active substance selected in the group consisting of carveol, thymol, eugenol, borneol, carvacrol, alpha-ionone, beta-ionone, and the isomers and derivatives and mixtures thereof, and at least one second container containing a second therapeutically active substance which is an antitumoral agent.

The invention further proposes a method for treating an affection due to a tumor characterized in that one administers, simultaneously or sequentially, to a patient having an affection due to a tumor, at least one first therapeutically active substance selected in the group consisting of carveol, thymol, eugenol, borneol, carvacrol, alpha-ionone, beta-ionone, and the isomers and derivatives and mixtures thereof, and at least one second therapeutically active substance which is an antitumoral agent.

Preferably, in said method, one simultaneously or sequentially administers to a patient having an affection due to a tumor between 10 and 200 mg/kg of body weight/day of said first therapeutically active substance, and between 2 and 100 mg/kg of body weight/day of second therapeutically active substance which is an antitumoral agent.

Preferably, in this method, said first therapeutically active substance is selected in the group consisting of carvacrol, eugenol and carveol and said second therapeutically active substance is doxorubicin.

The invention will be better understood and other aims and advantages thereof will appear more clearly in the explanatory description which follows.

The pharmaceutical composition according to the invention comprises as first therapeutically active substance thymol, eugenol, carvacrol, borneol, carveol, alpha-ionone, beta-ionone, and the derivatives and isomers as well as mixtures thereof.

The first therapeutically active substance must be pure.

These compounds themselves have well-known antitumoral properties.

Thymol, eugenol, carvacrol, borneol and carveol, alpha-ionone and beta-ionone are found in various proportions in different aromatic plant extracts, that is to say, they can be purified from such plants. However, they can quite simply be obtained by chemical synthesis.

As a matter of fact, the inventors have now discovered that said compounds have a potentiating effect on many therapeutically active substances including known antitumoral agents which are already used as medicaments specific in this field.

The second therapeutically active substance comprised in the pharmaceutical composition of the invention is therefore an antitumoral agent which is already known as such and already used as medicament specific in this field, and whose activity is potentiated.

Any other future antitumoral agents can also be used.

Examples of known antitumoral agents already used as medicaments specific in this field which can be used in the pharmaceutical composition of the invention, and whose effect will be potentiated by the first pure therapeutically active substance are folate antagonists, antimetabolites, alkylating agents, platinum salts, anthracyclin and intercalating agents, anti-topoisomerases and agents acting on cytoskeleton.

Methotrexate can be mentioned as folate antagonists, and 5-fluorouracil, fluorodeoxyuridine, cytosine arabinoside, 6-mercaptopurine and 6-thioguanine can be mentioned as antimetabolites.

Among alkylating agents used in the pharmaceutical composition of the invention, mechloroethamine, cyclophosphamide, ifosfamide, mephalan and chlorambucil can be mentioned.

Among aziridines, thiotepa, mitomycin C, aziridinylbenzoquinone (AZQ), alkylated sulfonates such as busulfan and nitrosoureas such as carmustine (BCNU), and lomustine (CCNU) and fotemustine can be mentioned.

Platinum salts used in the composition of the invention are cisplatinum and carboplatin.

Anthracyclin and intercalating agents used in the pharmaceutical composition of the invention are daunorubicin, doxorubicin (or adriamycin), epirubicin, dactinomycin (or actinomycin D), mitoxanthrone and amsacrine.

Among anti-topoisomerases which can be advantageously used in the pharmaceutical composition of the invention, tenoposide, etoposide, irinotecan and topotecan can be mentioned.

Among agents acting on cytoskeleton used in the pharmaceutical composition of the invention, vincristine, vinblastine, vindesine, vinorelbine, taxol®, taxotere® can be mentioned.

Other agents such as bleomycin and asparaginase can be advantageously used in the composition of the invention as second therapeutically active substance and antitumoral agent.

Particularly preferred is doxorubicin, more particularly in combination with carvacrol, thymol or carveol.

The second therapeutically active substance comprised in the pharmaceutical composition of the invention is therefore an antitumoral agent which is already known as such and whose activity is potentiated by the first therapeutically active substance.

Antitumoral agents used in the composition of the invention are folate antagonists, antimetabolites, alkylating agents, platinum salts, anthracyclin and intercalating agents, anti-topoisomerases and agents acting on cytoskeleton.

Methotrexate can be mentioned as folate antagonists, and 5-fluorouracil, fluorodeoxyuridine, cytosine arabinoside, 6-mercaptopurine and 6-thioguanine can be mentioned as antimetabolites.

Among alkylating agents used in the pharmaceutical composition of the invention, mechloroethamine, cyclophosphamide, ifosfamide, mephalan and chlorambucil can be mentioned.

Among aziridines, thiotepa, mitomycin C, aziridinylbenzoquinone (AZQ), alkylated sulfonates such as busulfan and nitrosoureas such as carmustine (BCNU), and lomustine (CCNU) and fotemustine can be mentioned.

Platinum salts used in the composition of the invention are cisplatinum and carboplatin.

Anthracyclin and intercalating agents used in the pharmaceutical composition of the invention are daunorubicin, doxorubicin (or adriamycin), epirubicin, dactinomycin (or actinomycin D), mitoxanthrone and amsacrine.

Said compounds can be used alone, or in combination with each other. The derivatives thereof, if they have antitumoral activity, can also be used.

Particularly preferred is doxorubicin, more particularly in combination with carvacrol, thymol or carveol.

Of course, the pharmaceutical composition according to the invention is not restricted to the use of only those antitumoral agents mentioned above. In fact, considering the potentiating effect exerted by the first therapeutically active substance defined in the invention, other known or future antitumoral agents can also be successfully used.

The pharmaceutical composition according to the invention can be formulated so as to be suitable for a simultaneous or sequential administration of said at least first and second therapeutically active substances.

The pharmaceutical form of the pharmaceutical composition of the invention shall be adapted to its use. For example, it can be used in the form of a solution, suspension, tablet or other. The compositions for parenteral administration are generally pharmaceutically acceptable sterile solutions or suspensions which can optionally be prepared immediately before use.

For the preparation of nonaqueous solutions or suspensions, it is possible to use natural vegetable oils like olive oil, sesame oil or paraffin oil or the injectable organic esters such as ethyl oleate. The sterile aqueous solutions can be composed of a solution of therapeutically active substances in water. The aqueous solutions are suitable for intravenous administration in so far as the pH is properly adjusted and/or they are made isotonic, for example by adding a sufficient amount of sodium chloride or glucose.

In fact, considering the chemical structure of antitumoral agents, and secondly, considering the chemical structure of carveol, carvacrol, thymol, eugenol, borneol, alpha-ionone and beta-ionone, it is thought, without intending to be bound by this theory, that carveol, carvacrol, thymol, eugenol, borneol, alpha-ionone and beta-ionone and the isomers, derivatives and mixtures thereof, interact with the antitumoral agents to form complexes having a structure which diffuses more easily into the body's physiological fluids and which diffuses more easily into the cytoplasm of targeted infected cells.

However, it has been shown that when the different components of the pharmaceutical composition of the invention are mixed in the presence of detergents such as Tween or Triton or solvents such as ethanol or DMSO (dimethyl sulfoxide), the active molecules of the first and second therapeutically active substance associate with the molecules of the detergents and solvents and do not form potentiating complexes.

Now it has been discovered that the potentiating complex forms when an aqueous agar suspension is used, as means of dispersion by viscosity.

Thus, the pharmaceutical composition of the invention will preferably be prepared without detergent and without solvent. For example, it will be prepared as an aqueous suspension made viscous by the addition of agar at a non-solidifying concentration, for example from 1 to 5 grams of agar per liter of suspension.

The pharmaceutical composition of the invention enables the treatment of local or general affections which withstand treatments by using doses of each of said first and second therapeutically active substance which are lower than the doses required for treating the same affections with one or the other of these same said first and second therapeutically active substances alone. In fact, the composition of the invention enables the use of doses of said first therapeutically active substance, when it is combined with said second therapeutically active substance, which are approximately two to five times lower than the doses required when said first therapeutically active substance is used alone, and of doses of said second therapeutically active substance, when it is combined with said first therapeutically active substance, which are two to five times lower than the doses required when said second therapeutically active substance is used alone.

The result is to offer a treatment which has the following advantages:

effective at very low doses against susceptible affections,

effective against affections resistant to a therapeutic agent,

effective against affections resistant to several therapeutic agents,

control of recurrence phenomena,

control of phenomena of resistant selection.

In all these cases, there is a reduction in the risks of toxicity and/or adverse effects, well known to the person of the art, thanks to potentiation which enables the administration of very low doses.

In addition, the costs of producing the treatment are reduced due to the use of small quantities of active substances used.

The pharmaceutical compositions according to the invention can be in the form of liposomes or associated with supports such as cyclodextrins or polyethylene glycols.

The pharmaceutical compositions of the invention are a simple and efficient means to combat the problems related to tumor affections in general which comprise mainly resistance to therapeutic agents and toxicity of the latter resulting from the use of high doses.

In fact, carveol, thymol, eugenol, borneol, carvacrol, alpha-ionone, beta-ionone and the derivatives, mixtures and isomers thereof, are simple molecules which have never been described as having any toxicity whatsoever and their addition with its potentiating effect on the second therapeutically active substance enables the use of much lower doses of said second therapeutically active substance.

In a first variant, then, the method for treating patients having a tumor affection consists in administering to said patients the dose, determined by the physician, of the pharmaceutical composition of the invention comprising suitable doses of at least one said first therapeutically active substance, combined with suitable doses of at least one said second therapeutically substance, that is, the suitable antitumoral agent.

In a second variant, the method for treating patients having a tumor affection consists in sequentially administering to said patients the dose determined by the physician of at least one said first therapeutically active substance, followed by the suitable dose of at least one said second therapeutically active substance, that is, the suitable antitumoral agent, or vice versa.

In this regard, the invention proposes a kit comprising at least one first container containing one of said first therapeutically active substances, and at least one second container containing one of said second therapeutically active substances.

Said kit enables health care personnel to prepare on demand either a mixture of suitable doses of the desired first therapeutic substance(s) and of the desired antitumoral agent(s), for a simultaneous administration, or to sequentially and separately administer the suitable dose of at least one said first therapeutically active substance, followed by the suitable dose of at least one said second therapeutically active substance, that is, the suitable antitumoral agent, or vice versa.

However, a mixture for simultaneous use shall be preferred in order to allow the potentiation complex to form and to act immediately after administration to the patient.

The invention will become clearer with the example below describing one embodiment and which are given for purposes of illustration and not by way of limitation.

### EXAMPLE 1

### Treatment of Tumor Cells with Doxorubicin

In vivo tests were carried out on HEP (human larynx carcinoma), BSR (hamster kidney carcinoma) and P815 (murin mastocytoma) cell lines cultivated on DMEM medium (Dulbecco's modified eagles medium) supplemented with 5% of fetal bovine serum (Gibco BRL, France), 1% of penicillin- streptomycin-neomycin, 0.2% of sodium bicarbonate. These percentages are given as ratio of the weight to the global volume of the medium.

In order to determine the concentration enables to induce a cytotoxicity for 50% of treated tumor cells, namely IC₅₀, in one hand for doxorubicin (adriamycin trade mark) alone, carvacrol alone, thymol alone and carveol alone, and in the other hand, for composition of the invention containing various concentrations of doxorubicin mixed with 0,012% (12 mg/100 mL of excipient solution) of either carvacrol orthymol, and 0,016% (16 mg/100 mL of excipient solution) of carveol.

In these compositions, excipient is agar solution.

The cytotoxic activity has been measured with the MTT method according to Mosmann T, 1983 (Rapid colorimetric assay for cellular growth and survival: application to proliferation and cytotoxicity assays. J. Immunol. Methods. 1983).

IC₅₀ have been determined. Obtained results are presented in Table 1 in which IC₅₀ are given in pg/mL of each said compositions for each cell lines.

TABLE 1

| Cell lines | HEP | BSR | P815 |
|---|---|---|---|
| Carvacrol | IC₅₀ = 0.03 | IC₅₀ = 0.03 | IC₅₀ = 0.03 |
| Thymol | IC₅₀ = 0.03 | IC₅₀ = 0.03 | IC₅₀ = 0.03 |
| Carveol | IC₅₀ = 0.04 | IC₅₀ = 0.04 | IC₅₀ = 0.04 |
| Doxorubicin | IC₅₀ = 20 | IC₅₀ = 25 | IC₅₀ = 15 |
| Doxorubicin + | IC₅₀ = 6 | IC₅₀ = 5 | IC₅₀ = 5 |
| 0.012% carvacrol | | | |
| Doxorubicin + | IC₅₀ = 6 | IC₅₀ = 6 | IC₅₀ = 5 |
| 0.012% thymol | | | |
| Doxorubicin + | IC₅₀ = 8 | IC₅₀ = 8 | IC₅₀ = 6 |
| 0.016% carveol | | | |

Table 1 shows that IC₅₀ of carvacrol alone and thymol alone is 0,03% (30 mg /100 mL), and that IC₅₀ of carveol alone is 0,04% (40 mg/100 mL excipient solution). Thus, in the tested compositions of the invention, carvacrol, thymol and carveol are at concentrations without any cytotoxic effects towards cell lines used in the assay.

Table 1 shows that IC₅₀ of doxorubicin used alone is between 15 and 25 μg/mL depending on the cell line.

Results presented in Table 1 clearly demonstrate that the composition according to the invention enables to induce a cytotoxic effect for 50% of tested tumor cells with a concentration of doxorubicin three times lower.

Thus, compositions of the invention exhibit a remarkable antitumoral activity in comparison with doxorubicin or thymol or carvacrol or carveol alone.

Moreover, an IC₅₀ of 8 μg/mL for doxorubicin does not exhibit cytotoxic activity towards cell lines used in the assay.

Similar results have been obtained with other families of known antitumoral agents by using eugenol, borneol, and the isomers and derivatives thereof, as potentiating agents.

Considering the chemical structure of antitumoral agents and the chemical structure of potentiating agents, the inventors think, without intending to be bound by this theory, that potentiating agents such as carvacrol, thymol, carveol, and derivatives and analogues thereof, interact with antitumoral agents to form complexes having a structure which diffuses more easily into the body's physiological fluids and which diffuses more easily into the cytoplasm of targeted tumor cells.

Of course, the invention is in no way restricted to the embodiments described herein which are given solely for purposes of illustration and not by way of limitation.

On the contrary, the invention comprises all the technical equivalents of the methods means described herein as well as the combinations thereof where such are carried out in the spirit of the invention.

1-12. (canceled)

13. A pharmaceutical composition comprising:
at least one first therapeutically active substance selected from the group consisting of carveol, thymol, eugenol, borneol, carvacrol, alpha-ionone, beta-ionone, and isomers, derivatives or mixtures thereof; and
at least one second therapeutically active substance which is an antitumoral agent.

14. The composition according to claim 13, wherein said at least one second therapeutically active substance is selected from the group consisting of folate antagonists, antimetabolites, alkylating agents, platinum salts, anthracyclin and intercalating agents, anti-topoisomerases, agents acting on cytoskeleton, bleomycin, asparaginase, and mixtures thereof.

15. The composition according to claim 14, wherein said at least one second therapeutically active substance is selected from the group consisting of methotrexate, 5-fluorouracil, fluorodeoxyuridine, cytosine arabinoside, 6-mercaptopurine, 6-thioguanine, mechloroethamine, cyclophosphamide, ifosfamide, melphalan, chlorambucil, thiotepa, mitomycin C, aziridinylbenzoquinone (AZQ), busulfan, carmustine (BCNU), lomustine (CCNU), fotemustine, carboplatin, daunorubicin, doxorubicin or adriamycin, epirubicin, dactinomycin or actinomycin D, mitoxanthrone, amsacrine, tenoposide, etoposide, irinotecan, topotecan, vincristine, vinblastine, vindesine, vinorelbine, taxol, taxotere, and mixtures thereof.

16. The composition according to claim 13, wherein said first therapeutically active substance is thymol, carveol or carvacrol and said at least one second therapeutically active substance is selected from the group consisting of folate antagonists, antimetabolites, alkylating agents, platinum salts, anthracyclin and intercalating agents, anti-topoisomerases, agents acting on cytoskeleton, bleomycin, asparaginase, and mixtures thereof.

17. The composition according to claim 13, wherein said first therapeutically active substance is thymol, carveol or carvacrol and said at least one second therapeutically active substance is selected from the group consisting of methotrexate, 5-fluorouracil, fluorodeoxyuridine, cytosine arabinoside, 6-mercaptopurine, 6-thioguanine, mechloroethamine, cyclophosphamide, ifosfamide, melphalan, chlorambucil, thiotepa, mitomycin C, aziridinylbenzoquinone (AZQ), busulfan, carmustine (BCNU), lomustine (CCNU), fotemustine, carboplatin, daunorubicin, doxorubicin or adriamycin, epirubicin, dactinomycin or actinomycin D, mitoxanthrone, amsacrine, tenoposide, etoposide, irinotecan, topotecan, vincristine, vinblastine, vindesine, vinorelbine, taxol, taxotere, and mixtures thereof.

18. The composition according to claim 17, said at least one second therapeutically active substance is doxorubicin.

19. The composition according to claim 14, wherein said first and second therapeutically active substances are suspended in an aqueous agar solution.

20. The composition according to claim 14, wherein said composition does not include any detergent or solvent.

21. A kit comprising:
at least one first container containing a first therapeutically active substance selected from the group consisting of carveol, thymol, eugenol, borneol, carvacrol, alpha-ionone, beta-ionone, and isomers, derivatives or mixtures thereof; and
at least one second container containing a second therapeutically active substance which is an antitumoral agent.

22. The kit according to claim 21, wherein said second therapeutically active substance is selected from the group consisting of folate antagonists, antimetabolites, alkylating agents, platinum salts, anthracyclin and intercalating agents, anti-topoisomerases, agents acting on cytoskeleton, bleomycin, asparaginase, and the mixtures thereof.

23. The kit according to claim 22, wherein said second therapeutically active substance is selected from the group consisting of methotrexate, 5-fluorouracil, fluorodeoxyuridine, cytosine arabinoside, 6-mercaptopurine, 6-thioguanine, mechloroethamine, cyclophosphamide, ifosfamide, melphalan, chlorambucil, thiotepa, mitomycin C, aziridinylbenzoquinone (AZQ), busulfan, carmustine (BCNU), lomustine (CCNU), fotemustine, carboplatin, daunorubicin, doxorubicin or adriamycin, epirubicin, dactinomycin or actinomycin D, mitoxanthrone, amsacrine, tenoposide, etoposide, irinotecan, topotecan, vincristine, vinblastine, vindesine, vinorelbine, taxol, taxotere, and mixtures thereof.

24. The kit according to claim 21, wherein said first therapeutically active substance is thymol, carveol or carvacrol and said second therapeutically active substance is selected from the group consisting of methotrexate, 5-fluorouracil, fluorodeoxyuridine, cytosine arabinoside, 6-mercaptopurine, 6-thioguanine, mechloroethamine, cyclophosphamide, ifosfamide, melphalan, chlorambucil, thiotepa, mitomycin C, aziridinylbenzoquinone (AZQ), busulfan, carmustine (BCNU), lomustine (CCNU), fotemustine, carboplatin, daunorubicin, doxorubicin or adriamycin, epirubicin, dactinomycin or actinomycin D, mitoxanthrone, amsacrine, tenoposide, etoposide, irinotecan, topotecan, vincristine, vinblastine, vindesine, vinorelbine, taxol, taxotere, and mixtures thereof.

25. The kit according to claim 24, wherein said therapeutically active substance is doxorubicin.

26. A method for treating a tumor comprising the simultaneous or sequential administration to a patient having a tumor:
at least one first therapeutically active substance selected from the group consisting of carveol, thymol, eugenol, borneol, carvacrol, alpha-ionone, beta-ionone and the isomers and derivatives and mixtures thereof; and
at least one second therapeutically active substance which is an antitumoral agent.

27. The method according to claim 26, wherein said at least one second therapeutically active substance is selected from the group consisting of folate antagonists, antimetabolites, alkylating agents, platinum salts, anthracyclin and intercalating agents, anti-topoisomerases, agents acting on cytoskeleton, bleomycin, asparaginase, and the mixtures thereof.

28. The method according to claim 27, wherein said at least one second therapeutically active substance is selected from the group consisting of methotrexate, 5-fluorouracil, fluorodeoxyuridine, cytosine arabinoside, 6-mercaptopurine, 6-thioguanine, mechloroethamine, cyclophosphamide, ifosfamide, melphalan, chlorambucil, thiotepa, mitomycin C, aziridinylbenzoquinone (AZQ), busulfan, carmustine (BCNU), lomustine (CCNU), fotemustine, carboplatin, daunorubicin, doxorubicin or adriamycin, epirubicin, dactinomycin or actinomycin D, mitoxanthrone, amsacrine, tenoposide, etoposide, irinotecan, topotecan, vincristine, vinblastine, vindesine, vinorelbine, taxol, taxotere, and mixtures thereof.

29. The method according to claim 26, wherein said at least one first therapeutically active substance is thymol, carveol or carvacrol and said at least one second therapeutically active substance is selected from the group consisting of methotrexate, 5-fluorouracil, fluorodeoxyuridine, cytosine arabinoside, 6-mercaptopurine, 6-thioguanine, mechloroethamine, cyclophosphamide, ifosfamide, melphalan, chlorambucil, thiotepa, mitomycin C, aziridinylbenzoquinone (AZQ), busulfan, carmustine (BCNU), lomustine (CCNU), fotemustine, carboplatin, daunorubicin, doxorubicin or adriamycin, epirubicin, dactinomycin or actinomycin D, mitoxanthrone, amsacrine, tenoposide, etoposide, irinotecan, topotecan, vincristine, vinblastine, vindesine, vinorelbine, taxol, taxotere, and mixtures thereof.

30. The method according to claim 29, wherein said at least one second therapeutically active substance is doxorubicin.

31. The method according to claim 26, wherein said method comprises the simultaneous or sequential administration of:
between 10 and 200 mg/kg of body weight/day of said first therapeutically active substance; and
between 2 and 100 mg/kg of body weight/day of said second therapeutically active substance.

32. The method according to claim 29, wherein said method comprises the simultaneous or sequential administration of:
between 10 and 200 mg/kg of body weight/day of said first therapeutically active substance; and
between 2 and 100 mg/kg of body weight/day of said second therapeutically active substance.
